(19) United States
(12) Patent Application Publication
(10) Pub. No.: US 2024/0216284 A1
(43) Pub. Date: Jul. 4, 2024

(54) ALPELISIB FORMULATION

(71) Applicant: Novartis AG, Basel (CH)

(72) Inventors: Laxman CHERKUPALLY, Hyderabad (IN); Sarah GOLD, Frick (CH); Charu KOCHHAR, Basel (CH); Muzammil TARIQ, Hyderabad (IN)

(21) Appl. No.: 18/558,415

(22) PCT Filed: Apr. 28, 2022
(86) PCT No.: PCT/IB2022/053968

§ 371 Date: Nov. 1, 2023

(30) Foreign Application Priority Data
May 3, 2021 (IN) 202111020206

(51) Int. Cl. A61K 9/50; A61K 31/4439

(52) U.S. Cl. CPC A61K 9/5042; A61K 9/5015; A61K 31/4439

(57) ABSTRACT

Alpelisib Formulation The invention features a granular formulation comprising an internal phase consisting of free flowing granules including alpelisib, or a pharmaceutically acceptable salt thereof, as active pharmaceutical ingredient and at least one pharmaceutically acceptable carrier material, and preferably in addition an external phase without said API comprising at least one pharmaceutically acceptable carrier material; and related invention embodiments.

### FIELD OF THE INVENTION

The present invention relates to a novel granular formulation comprising the compound (S)-pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), also known as (S)-N1-(4-methyl-5-(2-(1,1,1-trifluoro-2-methylpropan-2-yl)pyridin-4-yl)thiazol-2-yl)pyrrolidine-1,2-dicarboxamide, also known as BYL719 or alpelisib, or a pharmaceutically acceptable salt thereof, respectively, and its use or said granular formulation for use in the treatment of PIK3CA-Related Overgrowth Spectrum (PROS) and other related invention embodiments.

### BACKGROUND OF THE INVENTION

Tissue proliferation is a tightly regulated process in the organism from embryonic development to adult life. One of the main regulators of cell proliferation is the PI3K/AKT/mTOR signaling pathway. The hyper-activation of the PI3K/AKT/mTOR pathway results in significant dysregulation of cellular functions, which in turn leads to a competitive growth advantage. Somatic mutations and gains or losses in these genes are linked to many different solid and hematological tumors. In addition to the well-characterized role of PIK3CA in cancer, post zygotic somatic mutations in PIK3CA have also been identified in a spectrum of overgrowth disorders. These disorders comprise a wide group of clinically recognizable mutation-driven malformations referred to as PIK3CA-Related Overgrowth Spectrum (PROS). PROS is associated with overgrowth of tissues which include, but are not limited to, adipose, muscle, skin, bone, neural, blood or lymph vessels. PROS is characterized by congenital or early childhood-onset overgrowth, sporadic occurrence, and mosaic distribution. Segmental overgrowth is often congenital at onset, but it is usually noted by 1 year of age with progressive overgrowth of tissues persisting in some cases into adulthood. Complications of PROS depend on the anatomical site and extent of overgrowth, but may include functional impairment (for example, walking or swallowing), pain, cardiac function impairment, pulmonary hypertension, seizures, impaired neurological development, recurrent superficial infections, thromboembolism, and/or hemorrhage, amongst other manifestations all of which may be debilitating, and cause early mortality. Current treatment relies on surgery primarily with debulking objectives—amputation, and/or endovascular occlusive procedures. Regrowth following surgery frequently occurs and often requires repeated surgery. There is a large unmet need for targeted, new therapeutic approaches and effective treatments to combat PROS.

Alpelisib (BYL719) is approved in the United States (US FDA: 24 May 2019) to be used in combination with the endocrine therapy, fulvestrant, to treat postmenopausal women, and men, with hormone (HR)-positive, human epidermal growth factor receptor 2 (HER2)-negative, PIK3CA-mutated, advanced or metastatic breast cancer as detected by an FDA-approved test following progression on or after an endocrine-based regimen. In addition, alpelisib has been administered to patients suffering from severe clinical manifestations of PROS. Patients demonstrating substantial improvement were associated with partial or complete recovery from PROS-related complications. As a result of the positive effect of alpelisib, it became possible to avoid rescue surgery in a few patients when surgery was considered as an option before treatment start.

The formulation of the present invention is a novel, preservative-free, wet granulated product containing alpelisib or a pharmaceutically acceptable salt thereof in the form of granules, with a good dissolution profile and allowing tailored administration in patients that allows for easy, convenient and safe ingestion, at a pharmacologically active daily dosage, especially by the pediatric population.

### SUMMARY OF THE INVENTION

The present invention provides a granular formulation appropriate for administration to patients having difficulties in swallowing, geriatric patients and especially pediatric patients, comprising (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (or alpelisib or BYL719), or a pharmaceutically acceptable salt thereof, especially for use in the treatment of PIK3CA-Related Overgrowth Spectrum (PROS).

### DESCRIPTION OF PREFERRED EMBODIMENTS

The present invention, in a first embodiment, provides a free flowing (meaning it can be trickled) granular formulation, especially for the treatment of a patient having difficulty of swallowing, a geriatric patient or especially a pediatric patient, comprising an internal phase in the form of granules including (S)-pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide (also known as alpelisib; or as BYL719), or a pharmaceutically acceptable salt thereof, preferably alpelisib as the free base, as active pharmaceutical ingredient (API) and at least one pharmaceutically acceptable carrier material, and preferably in addition an external granular phase without said API comprising at least one pharmaceutically acceptable carrier material.

In a second embodiment, the invention provides a pharmaceutical single dosage unit receptacle comprising the granular formulation according to the first embodiment or as defined elsewhere herein.

In a third embodiment, the invention provides the granular formulation or the pharmaceutical single unit dosage receptacle according to the preceding embodiments or as defined elsewhere herein for use in the treatment of a human patient, especially any patient having difficulty swallowing, a geriatric patient or especially a pediatric patient, said use comprising the administration of said granular formulation in the treatment of (or to a patient suffering from) a proliferative disease, especially a mutation-driven malformations referred to as PIK3CA-Related Overgrowth Spectrum (PROS), preferably comprising a unit dosage from a pharmaceutical single dosage unit receptacle comprising the granular formulation according to the first invention embodiment or as defined elsewhere herein.

In a fourth embodiment, the invention provides the use of a granular formulation or the pharmaceutical single unit dosage receptacle according to the preceding first or second embodiment or as defined elsewhere herein in the treatment of a human patient, especially any patient having difficulty swallowing, a geriatric patient or especially a pediatric patient, said use comprising the administration of said granular formulation in the treatment of (or to a patient suffering from) a proliferative disease, especially a mutation-driven malformations referred to as PIK3CA-Related Overgrowth Spectrum (PROS), preferably comprising a unit dosage from a pharmaceutical single dosage unit receptacle comprising the granular formulation according to the first invention embodiment or as defined elsewhere herein.

In a fifth embodiment, the invention provides a method of treatment of a proliferative disease or especially a mutation-driven malformations referred to as PIK3CA-Related Overgrowth Spectrum (PROS), comprising administering, especially to a human patient in need of such treatment, especially any patient having difficulty swallowing, a geriatric patient or especially a pediatric patient, a therapeutically effective amount of a granular formulation as defined in the first embodiment or elsewhere herein, preferably using a pharmaceutical single dosage unit receptacle as defined in the second embodiment or elsewhere herein.

In a sixth embodiment, the invention provides the use of a granular formulation as defined in the first embodiment or elsewhere herein for the manufacture of a medicament including said granular formulation in the form of free flowing granules for use in the treatment of a proliferative disease, especially a mutation-driven malformation referred to as PIK3CA-Related Overgrowth Spectrum (PROS), especially for use in the manufacture of a pharmaceutical single unit dosage receptacle as defined in the second embodiment or elsewhere herein for said treatment.

In a seventh embodiment, the invention provides a process for manufacturing a granular formulation of the present invention, comprising forming a granulate having an internal phase and an external phase by:
(i.) Preparing, by wet granulating, an internal phase comprising (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (also named alpelisib), or a pharmaceutically acceptable salt thereof, especially the free base form of alpelisib, adding one or more diluents, adding a disintegrant, and adding a binder, and then drying the mixture;
(ii.) Preparing the external phase comprising a diluent and a lubricant; and preferably
(iii.) Filling a pharmaceutical single dosage unit receptacle with the resulting granular (dried) formulation.

In preferred embodiments of the first to seventh embodiment above and other embodiments herein, the granular formulation comprises the external phase which more preferably does not include a disintegrant, as this is not required to set the active pharmaceutical ingredient alpelisib free from the internal phase.

In another embodiment, particularly, in all embodiments the free flowing granular formulation, especially if packed in a pharmaceutical single dosage receptacle, in particular a stick pack, contains especially 1% to 5%, preferably 2 to 5%, such as 3% to 4%, e.g. 3.33%; or alternatively especially 20 mg to 200 mg, preferably 25 mg to 150 mg, such as 20 mg or 25 mg or 50 mg or 100 mg of alpelisib or a pharmaceutically acceptable salt thereof (then the amount mentioned refers to the alpelisib part of the salt in case of a salt of alpelisib), especially alpelisib in free form, in an or the internal phase including a diluent, especially lactose, sorbitol, or especially microcrystalline cellulose, such as Avicel PH101, especially in an amount of 25% to 45%, preferably form 28 to 42%, such as from 30 to 40%, e.g. 36.17%, and/or mannitol (especially Mannitol Pharma), especially in an amount of 25% to 45%, preferably from 28 to 42%, such as from 30 to 40%, e.g. 34%, a disintegrant, especially croscarmellose sodium, crospovidone or preferably sodium carboxymethyl starch, especially in an amount from 2 to 7%, preferably from 3 to 6%, such as from 3 to 7%, e.g. 5%, and a binder, especially hydroxypropyl cellulose, povidone, starch or preferably hydroxypropylmethyl cellulose, especially in an amount of 1 to 5%, preferably from 1.5 to 4.5%, such as from 2 to 4%, e.g. 3%,
where the external phase contains (or especially consists of) a diluent, especially lactose, sorbitol, mannitol or especially (preferably microcrystalline) cellulose, such as cellulose MK GR, especially in an amount of 10 to 30%, preferably from 10 to 25%, such as from 15 to 20%, e.g. 17.5%, and a lubricant, such as compritol 888, sodium stearyl fumarate or preferably magnesium stearate, especially in an amount of 0.25 to 4%, preferably from 0.3 to 3%, such as from 0.5 to 1.5%, e.g. 1%;
all amounts chosen such as to add up to 100% in the final granular formulation (the powder comprising the internal and if given external phase), respectively.

Where a percentage (%) of an ingredient is mentioned, this refers to the weight percentage (%) of the total granular formulation.

The embodiments with ranges of the same level of definition (level 1) without preference, level 2)=after “especially”, level 3)=after “preferably”, level 4)=after “such as”; level 5) after “e.g.”) define preferred embodiments of the ranges defined in the immediately preceding embodiment.

In a further embodiment, the free flowing granular formulation comprises an internal phase and an external phase packed in a stick pack pharmaceutical single dosage receptacle, wherein: the internal phase contains: (i) 1% to 5%, 2 to 5%, 3% to 4%, or 3.33% alpelisib or a pharmaceutically acceptable salt thereof; or alternatively 20 mg to 200 mg, 25 mg to 150 mg, 20 mg or 25 mg or 50 mg or 100 mg of alpelisib or a pharmaceutically acceptable salt thereof (then the amount mentioned refers to the alpelisib part of the salt in case of a salt of alpelisib), especially alpelisib in free form; and (ii) a diluent selected from lactose, sorbitol, and microcrystalline cellulose in an amount of 25% to 45%, 28 to 42%, 30 to 40%, or 36.17%, and/or mannitol in an amount of 25% to 45%, 28 to 42%, 30 to 40%, or 34%; (iii) a disintegrant selected from croscarmellose sodium, crospovidone and sodium carboxymethyl starch, in an amount from 2 to 7%, 3 to 6%, 3 to 7%, or 5%; and (iv) a binder selected from hydroxypropyl cellulose, povidone, starch and hydroxypropylmethyl cellulose, in an amount of 1 to 5%, 1.5 to 4.5%, 2 to 4%, or 3%; and wherein the external phase contains: (i) a diluent selected from lactose, sorbitol, mannitol and microcrystalline cellulose, in an amount of 10 to 30%, 10 to 25%, 15 to 20%, or 17.5%; and (ii) a lubricant selected from compritol 888, sodium stearyl fumarate and magnesium stearate, in an amount of 0.25 to 4%, 0.3 to 3%, 0.5 to 1.5%, or 1%; wherein all amounts chosen such as to add up to 100% in the final granular formulation.

In a further embodiment, the free flowing granular formulation comprises an internal phase and an external phase packed in a stick pack pharmaceutical single dosage receptacle, wherein: the internal phase contains: (i) 3% to 4%, or 3.33% alpelisib or a pharmaceutically acceptable salt thereof; or alternatively 20 mg or 25 mg or 50 mg or 100 mg of alpelisib or a pharmaceutically acceptable salt thereof (then the amount mentioned refers to the alpelisib part of the salt in case of a salt of alpelisib), especially alpelisib in free form; and (ii) a diluent selected from lactose, sorbitol, and microcrystalline cellulose in an amount of 30 to 40%, or 36.17%, and/or mannitol in an amount of 30 to 40%, or 34%; (iii) a disintegrant selected from croscarmellose sodium, crospovidone and sodium carboxymethyl starch, in an amount from 3 to 7%, or 5%; and (iv) a binder selected from hydroxypropyl cellulose, povidone, starch and hydroxypropylmethyl cellulose, in an amount of 2 to 4%, or 3%; and wherein the external phase contains: (i) a diluent selected from lactose, sorbitol, mannitol and microcrystalline cellulose, in an amount of 15 to 20%, or 17.5%; and (ii) a lubricant selected from compritol 888, sodium stearyl fumarate and magnesium stearate, in an amount of 0.5 to 1.5%, or 1%; wherein all amounts chosen such as to add up to 100% in the final granular formulation.

In a further embodiment, the free flowing granular formulation comprises an internal phase and an external phase packed in a stick pack pharmaceutical single dosage receptacle, wherein: the internal phase contains: (i) 3% to 4%, or 3.33% alpelisib or a pharmaceutically acceptable salt thereof; or alternatively 20 mg or 25 mg or 50 mg or 100 mg or 125 mg of alpelisib or a pharmaceutically acceptable salt thereof (then the amount mentioned refers to the alpelisib part of the salt in case of a salt of alpelisib), especially alpelisib in free form; and (ii) a diluent which is microcrystalline cellulose (such as Avicel PH101) in an amount of 30 to 40%, or 36.17%, and/or mannitol (such as Mannitol Pharma) in an amount of 30 to 40%, or 34%; (iii) a disintegrant which is sodium carboxymethyl starch, in an amount from 3 to 7%, or 5%; and (iv) a binder which is hydroxypropylmethyl cellulose, in an amount of 2 to 4%, or 3%; and wherein the external phase contains: (i) a diluent which is microcrystalline cellulose (such as cellulose MK GR), in an amount of 15 to 20%, or 17.5%; and (ii) a lubricant which is magnesium stearate, in an amount of 0.5 to 1.5%, or 1%; wherein all amounts chosen such as to add up to 100% in the final granular formulation.

In a further embodiment, the free flowing granular formulation comprises an internal phase and an external phase packed in a stick pack pharmaceutical single dosage receptacle, wherein: the internal phase contains: (i) 3% to 4%, or 3.33% alpelisib or a pharmaceutically acceptable salt thereof; or alternatively 20 mg or 25 mg or 50 mg or 100 mg of alpelisib or a pharmaceutically acceptable salt thereof (then the amount mentioned refers to the alpelisib part of the salt in case of a salt of alpelisib), especially alpelisib in free form; and (ii) a diluent which is microcrystalline cellulose (such as Avicel PH101) in an amount of 36.17%, and/or mannitol (such as Mannitol Pharma) in an amount of 34%; (iii) a disintegrant which is sodium carboxymethyl starch, in an amount of 5%; and (iv) a binder which is hydroxypropylmethyl cellulose, in an amount of 3%; and wherein the external phase contains: (i) a diluent which is microcrystalline cellulose (such as cellulose MK GR), in an amount of 17.5%; and (ii) a lubricant which is magnesium stearate, in an amount of 1%; wherein all amounts chosen such as to add up to 100% in the final granular formulation.

In one embodiment, the present invention relates to the free flowing granular formulation of the present invention for use in the treatment of a proliferative disease.

In one embodiment, the present invention relates to the free flowing granular formulation of the present invention for use in the treatment of PROS.

In one embodiment, the present invention relates to a method of treatment of a proliferative disease, preferably PROS, comprising administering to a patient in need thereof a free flowing granular formulation of the present invention comprising a therapeutically effective amount of (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (or alpelisib), or a pharmaceutically acceptable salt thereof.

In one embodiment, the present invention further relates to a pharmaceutical receptacle comprising a granular formulation according to the present invention and printed instructions directing the administration of a granular formulation of the invention containing (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (or alpelisib), or a pharmaceutically acceptable salt thereof.

### Definitions

Unless defined otherwise, all technical and scientific terms used herein have the same meaning as is commonly understood by one of skill in the art to which this invention belongs. The following general definitions shall apply in this specification, unless otherwise specified.

The term “alpelisib” means (S)-pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), or (S)-N1-(4-methyl-5-(2-(1,1,1-trifluoro-2-methylpropan-2-yl)pyridin-4-yl)thiazol-2-yl)pyrrolidine-1,2-dicarboxamide, or BYL719. Alpelisib and its pharmaceutically acceptable salts are described in PCT Patent Application No. WO2010/029082 (hereby incorporated by reference in its entirety). Methods for preparing alpelisib are described in Example 15, therein. Preferably, alpelisib is in the free base form (that is, not forming a salt with another different acid or base). Alpelisib, or a pharmaceutically acceptable salt thereof, especially alpelisib itself, is a mandatory active pharmaceutical ingredient (API) of the granular formulations according to the invention.

The terms “comprising” and “including” or “containing” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “pharmaceutically acceptable” refers to those compounds, materials, excipients, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

A “geriatric patient” preferably is a patient in the age of 60 or more years, e.g. in the age of 70 or more years.

A “pediatric patient” means patients that are less than 21 years, or 12 to 16 years (adolescents), or 2 to 12 years (children), or 1 month to 2 years (infants), or newborn to 1 month (neonates). Preferably pediatric patients are children or adolescent patient with an age below 21 years, 18 years or less, 15 years or less, 12 years or less, 9 years or less, 6 years or less, 5 years or less.

A “patient having difficulty of swallowing” is a patient who due to physical and/or psychologic restraints has difficulties of swallowing pharmaceutical formulations in the form of tablets, capsules or the like.

The term “(free flowing) granular formulation” (also referred to as “formulation of the invention”, “formulation of the present invention” or the like herein) refers to the fact that a formulation according to the invention is capable of free flowing in the dry state during dosing (though for administration to a patient it may be administered to a fluid, such as a juice, tea, coffee, soup, milk, yoghurt, water or other food or beverage).

The term “free flowing granules” especially refers to a dry powder consisting of the granules, so that the resulting powder is capable of free flowing in the dry state.

The term “internal phase” refers to the granulate phase including the active ingredient (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (alpelisib), or a pharmaceutically acceptable salt thereof, and at least one pharmaceutically acceptable carrier material, e.g. one or especially two diluents, one disintegrant and a binder.

The term “external phase” refers to the external phase of the granulate not comprising the API but only at least one pharmaceutically acceptable carrier material, e.g., containing a diluent and a lubricant.

The term “pharmaceutical single dosage unit receptacle” refers to any (preferably sealed after filling with the granular formulation) containment including one single dose, especially a flask (e.g. from metal, glass or plastics, or any combination thereof; preferably sealed after filling, e.g., with a lid or foil), a bottle (e.g. from metal, glass or plastics, or any combination thereof; preferably sealed after filling, e.g., with a lid or foil), a bag, e.g. made from sheet(s) of plastics (preferably metallized) and/or metal, a pouch, e.g. made from sheet(s) of plastics (preferably metallized) and/or metal, a sachet, e.g. from plastics (preferably metallized) or metal, or especially as a stick pack, where the bag, pouch, sachet or stick pack is preferably sealed, e.g. by thermal treatment, to form a tight receptacle.

The term “stick pack” refers to a highly preferred pharmaceutical single dosage unit receptacle in the embodiments of the present invention. Also referred to as “granules in a single-dose container”, the term can especially include or in particular refer to an aluminum sachet.

Receptacle enclosed formulations according to the invention, especially stick pack enclosed formulations, offer advantages for storage and transportation over, for example, suspension formulations for the pediatric population that are bulkier and more problematic to transport. A stick pack formulation does not require a dosing cup, spoon, bottle, dispenser or anything for measuring a dose. The preferred stick pack contains a granular formulation of the present invention embodiments and is, by comparison a small, single dose sachet in a robust, child resistant aluminum packaging.

The term “unit dosage” refers to a dosage amount to be administered to a patient during dosing.

The term “daily dosage” refers to the total dosage amount of the therapeutic agent administered to a specific patient in any single day or twenty-four hour period. Said daily dosage may preferably be administered by using one or more of the pharmaceutical single dosage unit receptacles according to the invention The phrase “effective amount” or “therapeutically effective amount” is preferably used herein to mean an amount sufficient to reduce, in particular by at least about 5 percent, especially by at least 15 percent, preferably by at least 50 percent, more preferably by at least 90 percent, and most preferably prevent, a clinically significant deficit in the activity, function and response of the subject in need thereof. Alternatively, an effective amount or therapeutically effective amount is an amount sufficient to provide an observable improvement over the baseline clinically observable signs and symptoms of a disease when compared with patients not treated.

Administration of a granular formulation of the invention can take place by direct application (from the receptacle or via e.g. a spoon or the like) into the mouth of the respective patient, or it can be made by pouring the granular formulation into a liquid or a food and then taking it up orally.

Where the term “treatment” is used, this especially means therapeutical treatment, be it with the goal of complete cure, partial cure, symptomatic relief or other forms of therapy or any other useful treatment.

The term “about” or “approximately” preferably shall have the meaning of within ±10%, more preferably within +5%, more preferably within +2% or ±1% of a given value or range.

(S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) is a specific 2-carboxamide cycloamino urea derivative compound that potently and selectively targets the (α)-isoform of class IA PI3K and having the following chemical structure:

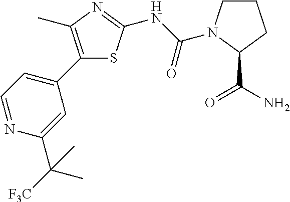 [SMILES: Cc1nc(NC(=O)N2CCC[C@H]2C(N)=O)sc1-c1ccnc(C(C)(C)C(F)(F)F)c1]

This compound is also known as (S)-N1-(4-methyl-5-(2-(1,1,1-trifluoro-2-methylpropan-2-yl)pyridin-4-yl)thiazol-2-yl)pyrrolidine-1,2-dicarboxamide or BYL719 or alpelisib.

(S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) and its pharmaceutically acceptable salts, are described in PCT Patent Application No. WO2010/029082, which is hereby incorporated by reference in its entirety, and methods of its preparation have been described, for example, in Example 15 therein. Preferably, alpelisib is in the free base form.

Pharmaceutically acceptable carrier materials suitable for use in the internal phase and/or the external phase of the granular formulation of the present invention include diluents and fillers, disintegrants, binders, and/or lubricants,

A diluent is preferably selected from the group consisting of mannitol (including the alpha, beta and delta polymorphs), sorbitol, malodextrin, lactose (e.g., lactose monohydrate), microcrystalline cellulose, maltitol, xylitol, and any combination thereof, Preferably, in the internal phase microcrystalline cellulose and/or mannitol is used, while in the external phase a cellulose, especially microcrystelline cellulose, is used.

A disintegrant is preferably selected from the group consisting of sodium starch glycolate, low-substituted hydroxypropyl cellulose, sodium croscarmellose, calcium croscarmellose, cross-linked polyvinylpyrrolidone (e.g., as commercially available under the tradenames Crospovidone® or Polyplasdone® or Kollidone®CL), cross-linked alginic acid, sodium alginate, potassium alginate, gellan gum, corn starch, pregelatinized starch, carboxymethylcellulose sodium, sodium carboxymethyl starchglycine and any combination thereof. Preferably, sodium carboxymethyl starch is used.

A binder is preferably selected from cellulose, such as, hydroxypropylmethyl cellulose, hydroxymethylcellulose, hydroxyethyl cellulose, starch, starch 1500, polyvinylpyrrolidone (povidone), and any combination thereof. Preferably, hydroxypropylmethyl cellulose is used.

A lubricant is preferably selected from sodium stearyl fumarate, magnesium stearate, stearic acid, hydrogenated castor oil, calcium stearate, aluminum stearate, PEG 4000-8000, talc, glyceryl monostearate, glyceryl dibehenate (e.g., commercially available by Gattefosse under trademark Compritol® 888 ATO), glyceryl palmito-stearic ester (e.g., commercially available by Gattefosse under trademark Precerol®), hydrogenated cotton seed oil, castor seed oil, and any combination thereof. Preferably, magnesium stearate is used.

Other pharmaceutically acceptable excipients for granular formulations (that can be trickled in the final packed state) are possible. Examples are pharmaceutically acceptable sweeteners, pharmaceutically acceptable coloring agents (dyes or pigments), pharmaceutically acceptable flavouring agents, preservatives, or the like, or mixtures of two or more thereof.

Formulations for geriatric patients and especially pediatric patients typically may contain preservatives and flavoring agents. The stick pack formulation of the present invention has no preservatives. In addition, alpelisib is a neutral molecule that does not have an unpleasant taste and therefore flavorings are not required in the granular formulation of the present invention.

### Pharmacology and Utility

The granular formulation of the present invention is useful for the treatment of proliferative diseases, especially a cancer or a PIK3CA mutation-driven malformations referred to as PIK3CA-Related Overgrowth Spectrum (PROS), preferably for treatment of patients having difficulties in swallowing, geriatric patients or pediatric patients, and especially pediatric patients. Examples of cancer suitable for treatment with the granular formulation of the present invention include, but are not limited to, breast cancer, ovarian cancer, head and neck cancer, pancreatic cancer, and colorectal cancer. Preferably, the proliferative disease treated with the granular formulation of the present invention is a cancer selected from the group consisting of breast cancer, ovarian cancer and head and neck cancer. Preferably, the proliferative disease treated with the granular formulation of the present invention is PROS.

Tissue proliferation is a tightly regulated process in the organism from embryonic development to adult life. One of the main regulators of cell proliferation is the PI3K/AKT/mTOR signaling pathway, which is a well-known target of multiple therapeutic strategies. The hyper-activation of the PI3K/AKT/mTOR pathway results in significant dysregulation of cellular functions, which in turn leads to a competitive growth advantage. Somatic mutations and gains or losses in these genes are linked to many different solid and hematological tumors (De Santis M C, et al., (2017) PI3K Signaling in Tissue Hyper-Proliferation: From Overgrowth Syndromes to Kidney Cysts. Cancers (Basel); 9(4):30). In addition to the well-characterized role of PIK3CA in cancer, post zygotic somatic mutations in PIK3CA have also been identified in a spectrum of overgrowth disorders comprising a wide group of clinically recognizable mutation-driven malformations referred to as PIK3CA-Related Overgrowth Spectrum (PROS). PIK3CA-Related Overgrowth Spectrum (PROS) designates a heterogeneous group of rare, asymmetric overgrowth disorders caused by post zygotic variants in the gene PIK3CA (Keppler-Noreuil K M, Sapp J C, Lindhurst M J, et al (2014) Clinical delineation and natural history of the PIK3CA-related overgrowth spectrum. Am. J. Med. Genet. A p. 1713-33). PIK3CA encodes the p110α catalytic subunit of phosphatidylinositol 3-kinase (PI3K), which transduces activation of tyrosine kinase growth factor and hormone receptors into activation of Protein Kinase B (AKT) and Mammalian Target of Rapamycin (mTOR) signaling to promote tissue growth. In PROS, activation of this pathway is associated with overgrowth which may include adipose tissue, muscle, skin, bone, blood or lymph vessels, or neural tissue (Kurek K C, et al., (2012) Somatic mosaic activating mutations in PIK3CA cause CLOVES syndrome. Am. J. Hum. Genet. 90(6):1108-15; Lindhurst M J, Parker V E, Payne F, et al (2012) Mosaic overgrowth with fibroadipose hyperplasia is caused by somatic activating mutations in PIK3CA. Nat. Genet; 44(8):928-33). The prevalence of PROS is difficult to estimate because of its rarity, its recent characterization (i.e., in 2014 by the National Institutes of Health), variation in ascertainment, the broad phenotypic spectrum, and the occurrence of atypical or mild phenotypes leading to misdiagnosis (Keppler-Noreuil K M, et al., (2014) Clinical delineation and natural history of the PIK3CA-related overgrowth spectrum. Am. J. Med. Genet. A p. 1713-33; Mirzaa G M, et al., (2013) Megalencephaly syndromes and activating mutations in the PI3K-AKT pathway: MPPH and MCAP. Am J Med Genet C Semin Med Genet p. 122-30). PROS is characterized by congenital or early childhood-onset overgrowth, sporadic occurrence, and mosaic distribution. Segmental overgrowth is often congenital at onset, but it is usually noted by 1 year of age with progressive overgrowth of tissues persisting in some cases into adulthood. Though some genotype-phenotype correlation in PROS has been suggested (Mirzaa G, et al., (2016) PIK3CA-associated developmental disorders exhibit distinct classes of mutations with variable expression and tissue distribution. JCI Insight;1(9): e87623; Keppler-Noreuil K M, et al., (2014) Clinical delineation and natural history of the PIK3CA-related overgrowth spectrum. Am. J. Med. Genet. A p. 1713-33.), the main determinant of phenotype is the timing and location of the pathogenic mutation. Consequently, PROS is characterized by a high degree of inter-individual phenotypic heterogeneity. The overgrowth features vary greatly for reasons which are unknown: some lesions exhibit excess growth which is limited to childhood, while other individuals have progressive soft tissue overgrowth during adult life. Complications of PROS depend on the anatomical site and extent of overgrowth, but may include functional impairment (for example, of walking or swallowing), pain, cardiac function impairment, pulmonary hypertension, seizures, impaired neurological development, recurrent superficial infections, thromboembolism, and/or hemorrhage, amongst other manifestations all of which may be debilitating, and cause early mortality. Current treatment relies on surgery primarily with debulking objectives—amputation, and/or endovascular occlusive procedures. Regrowth following surgery frequently occurs and often requires repeated surgery. There is large unmet need for targeted, new therapeutic approaches and effective treatments to combat PROS.

Alpelisib, which is also known as (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or BYL719), is an oral α-specific class I phosphatidylinositol-3-kinase (PI3K) inhibitor belonging to the 2-aminothiazole class of compounds. In biochemical assays, alpelisib potently inhibits the p110α subunit of PI3K (IC50=4.6 nM)≥50-fold compared to the other class I PI3K isoforms (e.g., p110β IC50=1156 nM, p110δ IC50=290 nM, p110γ IC50=250 nM), and is inactive against most other kinases (Fritsch C, et al., (2014) Characterization of the novel and specific PI3Kα inhibitor NVP-BYL719 and development of the patient stratification strategy for clinical trials. Mol. Cancer Ther., p. 1117-29).

Alpelisib has been administered to patients with PROS under a compassionate use program. It was administered to two patients (one adult and one child), who had confirmed PIK3CA mutations and suffered from severe clinical manifestations of PROS. The adult participant was a 29-year-old man who was previously treated with Sirolimus and progressed. The pediatric patient was a nine-year-old girl with confirmed PIK3CA c.3140A>G (H1047R) mutation who had not received any targeted systemic treatment for her PROS/CLOVES syndrome. Both patients tolerated treatment well and had no adverse effects with the exception of hyperglycemia which was experienced by the adult patient and was well controlled with nutrition therapy; alpelisib did not affect growth of the pediatric patient. In both patients shrinkage of volume of PROS lesions were associated with improved functions of affected organs and improved performance status. Several adult and pediatric patients with PROS have been successfully treated with alpelisib under compassionate use.

The granular formulation of the present invention is particularly useful for the treatment of PIK3CA-Related Overgrowth Spectrum (PROS) in pediatric patients.

In one embodiment, the granular formulation of the present invention can be used for the treatment of the PROS disorder fibroadipose hyperplasia (FH). While not malignant, this PROS disorder may cause health and medical issues such as problems walking due to the legs being a different length, problems moving a limb due to the overgrowth and challenges in regular daily activities due to deformity.

In another embodiment, the granular formulation of the present invention can be used for the treatment of Congenital, Lipomatous, Overgrowth, Vascular Malformations, Epidermal Nevi and Spinal/Skeletal Anomalies and/or Scoliosis, collectively CLOVES syndrome. CLOVES syndrome is a rare, progressive congenital disorder that involves multiple organs including the skin, the vascular system and the musculoskeletal system.

In another embodiment, the granular formulation of the present invention can be used for the treatment of megalencephaly-capillary malformation syndrome. Megalencephaly-capillary malformation syndrome is a rare developmental defect characterized by overgrowth of the brain and multiple somatic tissues, with capillary skin malformations, megalencephaly (MEG) or hemimegalencephaly (HMEG), cortical brain abnormalities such as polymicrogyria, facial dysmorphisms, abnormalities of somatic growth with asymmetry of the body and brain, developmental delay and digital anomalies.

In another embodiment, the granular formulation of the present invention can be used for the treatment of hemi hyperplasia-multiple lipomatosis syndrome. Hemi hyperplasia-multiple lipomatosis syndrome is a rare, genetic overgrowth syndrome characterized by non-progressive, asymmetrical, moderate hemi hyperplasia associated with slow growing, painless, multiple, recurrent, subcutaneous lipomatous masses distributed throughout entire body (in particular back, torso, extremities, fingers and axillae).

In another embodiment, the granular formulation of the present invention can be used for the treatment of hemimegalencephaly. Hemimegalencephaly is a rare malformation involving one side of the brain. Children with this disorder may have a large, asymmetrical head accompanied by seizures, partial paralysis, and impaired cognitive development often requiring hemispherectomy surgery.

In another embodiment, the granular formulation of the present invention can be used for the treatment of congenital infiltrating lipomatosis of the face (CILF). A very rare disorder in which mature lipocytes invade into adjacent tissues in the facial region resulting in facial asymmetry.

In a further embodiment, the present invention relates to a method of treatment of a PIK3CA-Related Overgrowth Spectrum (PROS) disorder comprising administering to a patient in need thereof a granular formulation of the present invention comprising a therapeutically effective amount of alpelisib or a pharmaceutically acceptable salt thereof.

The activity and characteristics of the granular formulation of the present invention may be indicated in standard clinical trials and/or animal trials.

In adult patients (e.g. geriatric patients or other adult patients having swallowing difficulties), alpelisib may be orally administered at an effective daily dose of about 1 to 6.5 mg/kg in human adults. Alpelisib may be orally administered to a 50 to 70 kg body weight human adult at a daily dosage of about 70 mg to 455 mg, for example, about 100 to 400 mg, or about 240 mg to 400 mg, or about 125 mg to 400 mg, or about 125 mg to 300 mg, in a single dose or in divided doses up to four times a day. Preferably, alpelisib is administered to a 50 to 70 kg body weight human adult (e.g. a geriatric patient or an adult with swallowing difficulties) at a daily dosage of about of 125, 200, 250 or 300 mg/day, or 350 mg to about 400 mg. In adult patients, alpelisib is preferably administered at 250 mg orally once daily with food.

In pediatric patients, alpelisib may be administered orally at an effective daily dosage, per patient, of about 10 mg per day, or 20 mg per day (preferred), or 25 mg/day (preferred), or 30 mg per day, or 40 mg per day, or 50 mg per day (preferred), or 60 mg per day, or 70 mg per day, or 75 mg/day, or 80 mg per day, or 90 mg per day, or 100 mg per day, or 110 mg per day, or 120 mg per day, or 125 mg/day (preferred), or 130 mg per day, 140 mg per day, or 150 mg per day, or 175 mg/day or 200 mg/day (preferred) or 250 mg/day. In pediatric patients, alpelisib is preferably administered at 50 mg orally once daily with food.

It will be understood that the specific dose level for any particular patient will depend on a variety of factors including the age, body weight, general health, drug combination with one or more active drugs, type and severity of the disease. With the teachings of the present invention, one skilled in the art would have the experience and skill to select the proper dose level treatment of a specific patient.

In another embodiment, the present invention further relates to a pharmaceutical single dosage unit receptacle comprising a granular formulation according to the present invention and printed instructions directing the administration of alpelisib, or a pharmaceutically acceptable salt thereof, for the treatment of PROS.

### EXAMPLES

The following Example illustrates the invention described above, but is not, however, intended to limit the scope of the invention in any way.

### Example 1

### Manufacture of 20 mg or 50 mg Granular Formulation

The 20 mg and 50 mg granular formulation was manufactured using the unit amounts in Table 1, below. The environmental conditions for the manufacturing process were 22° C.±3° C., 55% RH±10% RH.

Mannitol Pharma, alpelisib, natriumcarboxymethylstarke (NA-carboxymethyl-starch) and Avicel PH101 (cellulose, microcrystalline PH101) were blended together in a high shear granulator. HPMC-603 (HPMC, hypromellose) was dissolved in purified water in a stainless steel vessel with stirring. Swelling and defoaming were permitted (for about 5 hours) until a clear solution was obtained. The dissolved HPMC-603 was then added to the mixture in the high shear granulator at a speed of 0.8 kg/min-1.0 kg/min while granulating at the impeller speed of 130-203 rpm and chopper speed of 1450 rpm. The liquid feeding system was rinsed with the required amount of water needed to meet the target water addition amount at a speed of 0.8 kg/min-1.0 kg/min. The wet mass was kneaded in the high shear granulator for 10-60 seconds at an impeller speed of 150-203 rpm and chopper speed of 1450 rpm. The granulate was dried in a fluid bed dryer until loss of drying (% LOD) of ca.≤1.7% is achieved. The dried granulate was screened in a screening mill (screen size 1 mm, round wire). The external phase excipient cellulose MK GR (PH102) was sieved through a screening mill (screen size 1 mm/round wire), added to the granulate and blended in a blender at a target speed of 9 rpm for 18 minutes. Magnesium stearate was sieved (0.5 mm) and added to the granulate followed by blending in a blender at the target speed of 9 rpm for 11 minutes. The final blend was ready and used for filling using the packaging material into stick packs (stick pack dimensions are 90 mm×25 mm for both strengths). The holding time of the final blend was 30 days and storage temperature did not exceed 25° C.

TABLE 1

| | | | | |
|---|---|---|---|---|
| | | Composition | Composition | |
| | | per | per | |
| | | dosage unit | dosage unit | |
| | | (mg) | (mg) | Composition |
| | | 20 mg | 50 mg | per dosage |
| Component | Function | strength | strength | unit (%) |

Internal Phase

| | | | | |
|---|---|---|---|---|
| Alpelisib | Drug | 20.00 | 50.00 | 3.33 |
| | substance | | | |
| Avicel PH101 | Diluent | 217.0 | 542.50 | 36.17 |
| Mannitol | Diluent | 204.00 | 510.00 | 34.00 |
| Pharma | | | | |
| sodium | Disintegrant | 30.00 | 75.00 | 5.00 |
| carboxymethyl | | | | |
| starch | | | | |
| HPMC-603 | Binder | 18.00 | 45.00 | 3.00 |
| Water | | | | 34-36 |
| (deionized) | | | | |

External Phase

| | | | | |
|---|---|---|---|---|
| Cellulose | Diluent | 105.00 | 262.50 | 17.50 |
| MK GR* | | | | |
| Magnesium | Lubricant | 6.00 | 15.00 | 1.00 |
| stearate | | | | |

The granular formulation of the present invention has good flowability resulting in uniform filling of the receptacles, such as stick packs. A characteristic of the granular formulation of the present invention is the reduced percentage of fines (amount of powder remaining in the granule) which helps avoid dust generation with fine particles settling on the sealing area of a stick pack packaging, interfering with the stick pack sealing.

1. A free flowing granular formulation comprising an internal phase in the form of granules including alpelisib, or a pharmaceutically acceptable salt thereof, as active pharmaceutical ingredient and at least one pharmaceutically acceptable carrier material, and in addition an external granular phase without said active pharmaceutical ingredient comprising at least one pharmaceutically acceptable carrier material.

2. (canceled)

3. (canceled)

4. The free flowing granular formulation according claim 1, comprising an internal phase and an external phase packed in a stick pack pharmaceutical single dosage receptacle, wherein: the internal phase contains: (i) 1% to 5%, 2 to 5%, 3% to 4%, or 3.33% alpelisib or a pharmaceutically acceptable salt thereof; or alternatively 20 mg to 200 mg, 25 mg to 150 mg, 20 mg or 25 mg or 50 mg or 100 mg or 125 mg of alpelisib or a pharmaceutically acceptable salt thereof; and (ii) a diluent selected from lactose, sorbitol, and microcrystalline cellulose in an amount of 25% to 45%, 28 to 42%, 30 to 40%, or 36.17%, and/or mannitol in an amount of 25% to 45%, 28 to 42%, 30 to 40%, or 34%; (iii) a disintegrant selected from croscarmellose sodium, crospovidone and sodium carboxymethyl starch, in an amount from 2 to 7%, 3 to 6%, 3 to 7%, or 5%;
and (iv) a binder selected from hydroxypropyl cellulose, povidone, starch and hydroxypropylmethyl cellulose, in an amount of 1 to 5%, 1.5 to 4.5%, 2 to 4%, or 3%; and
wherein the external phase contains: (i) a diluent selected from lactose, sorbitol, mannitol and microcrystalline cellulose, in an amount of 10 to 30%, 10 to 25%, 15 to 20%, or 17.5%; and (ii) a lubricant selected from compritol 888, sodium stearyl fumarate and magnesium stearate, in an amount of 0.25 to 4%, 0.3 to 3%, 0.5 to 1.5%, or 1%; wherein all amounts chosen such as to add up to 100% in the final granular formulation.

5. The free flowing granular formulation according to claim 1, comprising an internal phase and an external phase packed in a stick pack pharmaceutical single dosage receptacle, wherein: the internal phase contains: (i) 3% to 4%, or 3.33% alpelisib or a pharmaceutically acceptable salt thereof; or alternatively 20 mg or 25 mg or 50 mg or 100 mg of alpelisib or a pharmaceutically acceptable salt thereof;
and (ii) a diluent selected from lactose, sorbitol, and microcrystalline cellulose in an amount of 30 to 40%, or 36.17%, and/or mannitol in an amount of 30 to 40%, or 34%; (iii) a disintegrant selected from croscarmellose sodium, crospovidone and sodium carboxymethyl starch, in an amount from 3 to 7%, or 5%; and (iv) a binder selected from hydroxypropyl cellulose, povidone, starch and hydroxypropylmethyl cellulose, in an amount of 2 to 4%, or 3%;
and wherein the external phase contains: (i) a diluent selected from lactose, sorbitol, mannitol and microcrystalline cellulose, in an amount of 15 to 20%, or 17.5%; and (ii) a lubricant selected from compritol 888, sodium stearyl fumarate and magnesium stearate, in an amount of 0.5 to 1.5%, or 1%;
wherein all amounts chosen such as to add up to 100% in the final granular formulation.

6. The free flowing granular formulation according to claim 1, comprising an internal phase and an external phase packed in a stick pack pharmaceutical single dosage receptacle, wherein: the internal phase contains: (i) 3% to 4%, or 3.33% alpelisib or a pharmaceutically acceptable salt thereof; or alternatively 20 mg or 25 mg or 50 mg or 100 mg of alpelisib or a pharmaceutically acceptable salt thereof;
and (ii) a diluent which is microcrystalline cellulose (such as Avicel PH101) in an amount of 30 to 40%, or 36.17%, and/or mannitol (such as Mannitol Pharma) in an amount of 30 to 40%, or 34%; (iii) a disintegrant which is sodium carboxymethyl starch, in an amount from 3 to 7%, or 5%; and (iv) a binder which is hydroxypropylmethyl cellulose, in an amount of 2 to 4%, or 3%; and wherein the external phase contains: (i) a diluent which is microcrystalline cellulose (such as cellulose MK GR), in an amount of 15 to 20%, or 17.5%; and (ii) a lubricant which is magnesium stearate, in an amount of 0.5 to 1.5%, or 1%; wherein all amounts chosen such as to add up to 100% in the final granular formulation.

7. The free flowing granular formulation according to claim 1, comprising an internal phase and an external phase packed in a stick pack pharmaceutical single dosage receptacle, wherein: the internal phase contains: (i) 3% to 4%, or 3.33% alpelisib or a pharmaceutically acceptable salt thereof; or alternatively 20 mg or 25 mg or 50 mg or 100 mg of alpelisib or a pharmaceutically acceptable salt thereof;
and (ii) a diluent which is microcrystalline cellulose (such as Avicel PH101) in an amount of 36.17%, and/or mannitol (such as Mannitol Pharma) in an amount of 34%; (iii) a disintegrant which is sodium carboxymethyl starch, in an amount of 5%; and (iv) a binder which is hydroxypropylmethyl cellulose, in an amount of 3%; and wherein the external phase contains: (i) a diluent which is microcrystalline cellulose (such as cellulose MK GR), in an amount of 17.5%; and (ii) a lubricant which is magnesium stearate, in an amount of 1%; wherein all amounts chosen such as to add up to 100% in the final granular formulation.

8. A method for the treatment of a human patient having difficulty swallowing, selected from a geriatric patient or a pediatric patient, said method comprising the administration of the granular formulation of claim 1 in the treatment of a proliferative disease involving mutation-driven malformations related to PIK3CA-Related Overgrowth Spectrum (PROS), the method further comprising wherein the granular formulation is administered in a unit dosage from a stick pack pharmaceutical single dosage unit receptacle comprising the granular formulation to said patient.

9. A method for the treatment of a pediatric patient having difficulty swallowing said method comprising the administration of the granular formulation of claim 1 in the treatment of a proliferative disease involving mutation-driven malformations related to PIK3CA-Related Overgrowth Spectrum (PROS).

10. The method according to claim 9, wherein the granular formulation is administered using a stick pack pharmaceutical single dosage unit receptacle comprising the granular formulation of claim 4.

11. A method of treatment of a proliferative disease or a mutation driven malformation referred to as PROS, comprising administering to a human patient in need of such treatment having difficulty in swallowing, selected from a geriatric patient or a pediatric patient, of a therapeutically effective amount of a granular formulation according to claim 1.

12. The method of claim 11, where the granular formulation is administered using a pharmaceutical single dosage unit receptacle as defined in claim 4.

13. (canceled)

14. (canceled)

15. A process for manufacturing a granular formulation according to claim 1, comprising forming a granulate having an internal phase and an external phase by:
(i) Preparing by wet granulating an internal phase comprising alpelisib, or a pharmaceutically acceptable salt thereof, adding one or more diluents, adding a disintegrant, and adding a binder, and then drying the mixture; and
(ii) Preparing the external phase comprising a diluent and a lubricant.

16. A process according to claim 15, further comprising, in a step (iii), filling a stick pack pharmaceutical single dosage unit receptacle with the resulting dried granular formulation.

17. A method of treating a patient having difficulty swallowing, the method comprising administering to the patient the free flowing granular formulation of claim 1, wherein the patient is selected from a geriatric patient and a pediatric patient.

18. The method of claim 17, wherein the patient is a pediatric patient.

19. A method of treatment of a proliferative disease or a mutation driven malformation referred to as PROS, comprising administering to a human patient in need of such treatment having difficulty in swallowing, selected from a geriatric patient or a pediatric patient, of a therapeutically effective amount of a granular formulation according to claim 4.

20. A method of treatment of a proliferative disease or a mutation driven malformation referred to as PROS, comprising administering to a human patient in need of such treatment having difficulty in swallowing, selected from a geriatric patient or a pediatric patient, of a therapeutically effective amount of a granular formulation according to claim 5.

21. A method of treatment of a proliferative disease or a mutation driven malformation referred to as PROS, comprising administering to a human patient in need of such treatment having difficulty in swallowing, selected from a geriatric patient or a pediatric patient, of a therapeutically effective amount of a granular formulation according to claim 6.

22. A method of treatment of a proliferative disease or a mutation driven malformation referred to as PROS, comprising administering to a human patient in need of such treatment having difficulty in swallowing, selected from a geriatric patient or a pediatric patient, of a therapeutically effective amount of a granular formulation according to claim 7.
